(19) United States
(12) Patent Application Publication
(10) Pub. No.: US 2015/0197574 A1
(43) Pub. Date: Jul. 16, 2015

(54) USE OF TRANSFERRIN RECEPTOR ANTAGONIST FOR THE TREATMENT OF THALASSEMIA

(71) Applicants: INSERM (INSTITUT NATIONAL DE LA SANTE ET DE LA RECHERCHE MEDICALE), Paris (FR); CENTRE NATIONAL DE LA RECHERCHE SCIENTIQUE OF THALASSEMIA, Paris (FR); UNIVERSITE PARIS DESCARTES, Paris (FR); FONDATION IMAGE, Paris (FR); UNIVERSITE PARIS-SUD XI, Orsay Cedex (FR); ASSISTANCE PUBLIQUE-HOPITAUX DE PARIS (APHP), Paris (FR); UNIVERSITE PARIS DIDEROT - PARIS 7, Paris (FR)

(72) Inventors: Ivan Cruz-Moura, Paris (FR); Olivier Hermine, Paris (FR); Michael Dussiot, Paris (FR); Etienne Paubelle, Paris (FR); Thiago Trovati Maciel, Paris (FR)

(21) Appl. No.: 14/418,457

(22) PCT Filed: Aug. 2, 2013
(86) PCT No.: PCT/EP2013/066253

§ 371 Date: Jan. 30, 2015

(30) Foreign Application Priority Data
Aug. 2, 2012 (EP) 12305955.2

(51) Int. Cl. C07K 16/28

(52) U.S. Cl. CPC C07K 16/2881; C07K 2317/24; C07K 2317/76; A61K 2039/505

(57) ABSTRACT

The present disclosure relates to antagonists of transferrin receptor and compositions and methods of use of said antagonists for treating pathological disorders such as thalassemia disorders

### FIELD OF THE INVENTION

This invention is in the fields of biology and immunotherapy. It concern methods of treating thalassemias with transferrin receptor (TfR1) antagonist. More specifically, it concerns use of anti-TfR1 antibodies or an antigen-binding portions of said antibodies, for the treatment of β thalassemia.

### BACKGROUND OF THE INVENTION

Late-stage erythropoises is largely committed to the production of the oxygen carrier hemoglobin (Hb), a tetrameric protein consisting of two α-globin and two β-globin subunits. β-thalassemia, is a common inherited hemoglobinopathy characterized by impaired or absent β-globin gene production with consequent accumulation of unpaired α-subunits[1]. The excess of unbound free α-globin precipitate in maturing erythroid cells and induces the production of reactive oxygen species (ROS) resulting in cellular oxidative stress damage [2,3]. The presence of α-globin precipitates is also associated to a reduced red blood cell (RBC) half-life and to the clinical features of β-thalassemia highlighting its importance in the pathogenesis of the disease[4].

Ineffective erythropoiesis (IE) is a hallmark of the β-thalassemia and is characterized by an accelerated proliferation and differentiation of early erythroid progenitors associated to the increased apoptosis of the maturing nucleated erythroid cells. This phenomenon results in anemia and is accompanied by compensatory extramedullary erythropoiesis leading to a hepatosplenomegaly.

Another feature of the disease is systemic iron overload which can be aggravated by the necessity of blood transfusions in severe forms of disease (β-thalassemia major or Cooley anemia) and necessary to reduce the anemia. β-thalassemia intermedia patients usually present increased gastrointestinal iron absorption, decreased circulating levels of the hypoferrimia-related hormone hepcidin and tissue iron overload which also contribute to disease pathology [5].

The current standard of care for treating diseases associated with inefficient erythropoiesis (IE) includes red blood cell (RBC) transfusions and iron chelation therapy. However, there are many downsides that accompany these current treatment methods, such as the risk of infection, development of red blood cell antibodies, iron overload, splenomegaly, and cost. Gargenghi S. et al[6] suggest use of hepcidin for the treatment of β-thalassemia. Recently, Li et al. [7] suggest use of transferin for the treatment of β-thalassemia.

Accordingly, there is a need for a new therapeutic strategy that simultaneously improves the efficiency of erythropoiesis, to restore haemoglobin levels and chelates iron from storage in the liver, spleen and heart of a subject without such unwanted side effects.

The inventors show that blocking the binding of Transferin to TfR1 constitutes an alternative therapeutic axis in β-thalassemia.

### SUMMARY OF THE INVENTION

The present invention therefore provides isolated antagonist of transferrin receptor (TfR1), for a novel use in the treatment of thalassemia disorders, more particularly in treating β-thalassemia.

### DETAILED DESCRIPTION OF THE INVENTION

The inventors show here that impairing iron-loaded transferrin uptake by treatment with an anti-TfR1 monoclonal antibody [8] resulted in decreased IE and iron orverload in an experimental model of β-thalassemia intermedia (Hbbth1/th1 mice [9]). Treated mice presented decreased splenomegaly with a reduced accumulation of polychromatophil erythroblasts and a normalization of serum bilirrubin levels. Blocking iron-loaded transferrin uptake also resulted in decreased serum iron and transferrin saturation and increased serum transferrin levels. Treated mice also partially restored hemoglobin levels and Mean Corpuscular Hemoglobin (MCH) and MCH concentration (MCHC). Therefore, the inventors propose that blocking TfR1 function with an antagonist of TfR1 is an alternative therapeutic axis in the treatment of thalassemia.

Accordingly, the present invention relates to an antagonist of TfR1 for use in the treatment of thalassemia.

Another aspect of the invention relates to a in vivo method for treating thalassemia, comprising administering to a subject in need thereof a therapeutically effective amount of an antagonist of TfR1.

### DEFINITIONS

Throughout the specification, several terms are employed and are defined in the following paragraphs.

The term “Thalassamia” also called “Thalassaemia” or “Thalassaemia disorders” or “Thalassamia disorders” refers to a group of inherited autosomal recessive blood disorders that originated in the Mediterranean region. In thalassemia the genetic defect, which could be either mutation or deletion, results in reduced rate of synthesis or no synthesis of one of the globin chains that make up hemoglobin. This can cause the formation of abnormal hemoglobin molecules, thus causing anemia, the characteristic presenting symptom of the thalassemias. The two major forms of the disorder are alpha- and beta-thalassamia. “Beta-thalassamia” or “β-thalassemia”, is a common inherited hemoglobinopathy characterized by impaired or absent β-globin gene production with consequent accumulation of unpaired α-subunits[1]. The excess of unbound free α-globin precipitate in maturing erythroid cells and induces the production of reactive oxygen species (ROS) resulting in cellular oxidative stress damage [2,3]. The presence of α-globin precipitates is also associated to a reduced RBC half-life and to the clinical features of β-thalassemia highlighting its importance in the pathogenesis of the disease[4]. “Alpha-thalassemia” or “α-thalassemia” is a form of thalassemia involving the genes HBA1 and HBA2. Alpha-thalassemia is due to impaired production of 1, 2, 3, or 4 alpha globin chains, leading to a relative excess of beta globin chains. The degree of impairment is based on which clinical phenotype is present (how many chains are affected).

The term “TfR1” has its general meaning in the art and refers to Transferrin receptor 1. Transferrin receptor 1 (CD71/TfR1) is an evolutionary conserved receptor [10,11] implicated in cellular iron uptake through its binding to transferrin, the serum iron transporter.

As used herein, the term “antagonists of TfR1” is intended to refer to any agent which competitively inhibits binding of the transferrin to transferrin receptor (TfR1). In a preferred embodiment, the antagonist specifically binds to TfR1 in a sufficient manner to compete with transferin for the binding to TfR1. Inhibition of binding of the transferrin to transferrin receptor may be determined by any competing assays well known in the art. For example the assay may consist in determining the ability of the agent to be tested as an antagonist of TfR1 to bind to the TfR (preferably expressed at the surface of a cell). The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. A method for determining the KD of an antibody is by using surface plasmon resonance, or using a biosensor system such as a Biacore® system. In specific embodiments, an antagonist that “specifically binds to TfR1” is intended to refer to an antagonist that binds to human TfR1 polypeptide with a KD of 104 or less, 100 nM or less, 10 nM or less, or 3 nM or less. Then a competitive assay may be settled to determine the ability of the agent to inhibit the binding of transferin to TfR1. The assay may typically comprise i) contacting TfR1 expressing cell with the agent to be tested with transferin (e.g. a labelled transferin) ii) determining the level of internalization of transferin into the cell iii) comparing the level determined at step i) with the level determined in the absence of the agent to be tested and iv) positively selecting the agent when the level determined in step i) is lower that the level determined in the absence of the agent to be tested. Other functional assays may also be envisaged such prevention of the iron-loaded transferrin uptake, like transferrin uptake assays which could be also used to evaluate the ability of transferrin receptor antagonists to block iron-loaded transferrin internalization [see 12,13]. Preferably, inhibition in the presence of the antagonist must be observed in a dose-dependent manner and the measured signal is at least 10% lower, preferably at least 50% lower than the signal measured with a negative control under comparable conditions. Preferably, the antagonist according to the invention exhibits an IC50 of at least 1 μM, preferably 100 nM as measured in at least one of the assays described above.

According to the invention, the antagonists of TfR1 provide the following technical advantages for the treatment of β-thalassemia: decrease inefficient erythropoiesis, reduce splenomegaly, increase the transferrin synthesis, decrease serum iron overload without inducing anemia and reduce transferrin saturation level and increase the amount of hemoglobin/red blood cell.

In particular embodiment, the antagonist of TfR1 is selected from the group consisting of antibodies, antigen-binding portions of antibodies, small organic molecules, aptamers or polypeptides. In a preferred embodiment the antagonist of TfR1 is an anti-TfR1 antibody or an antigen-binding portion thereof.

The term “antibody” has its general meaning in the art. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., the ligand binding domain of transferrin receptor). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a F(ab′)2 fragment, a Fd fragment consisting of the VH and CH1 domains; a Fv fragment consisting of the VL and VH domains of a single arm of an antibody; a dAb fragment (Ward et al., 1989 Nature 341:544-546), which consists of a soluble VH domain, or any fusion proteins comprising such antigen-binding portion.

Furthermore, although the two domains of the Fv fragment, VL and VH, are coded for by separate genes, they can be joined, using recombinant methods, by a synthetic linker that enables them to be made as a single chain protein in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

The terms “monoclonal antibody” or “monoclonal antibody composition” as used herein refer to a preparation of antibody molecules of unique amino acid sequence structure for the variable regions. A monoclonal antibody composition therefore displays a single binding specificity and affinity for a particular epitope.

The term “humanized antibody”, as used herein, is intended to include antibodies that contain minimal sequence derived from non-human immunoglobulin sequences. For the most part, humanized antibodies are human immunoglobulins (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity and capacity. The phrase “complementarity determining region” refers to amino acid sequences which together define the binding affinity and specificity of the natural Fv region of a native immunoglobulin binding site. See, e.g. Chothia et al (1987) J. Mol. Biol. 196:901-917: Kabat et al (1991) US Dept. of Health and Human Services, NIH Publication No. 91-3242). The phrase “constant region” refers to the portion of the antibody molecule that confers effector functions. In previous work, directed towards producing non-immunogenic antibodies for use in therapy of human disease, mouse constant regions were substituted by human constant regions. The constant regions of the subject humanized antibodies were derived from human immunoglobulins. Humanization can be performed following the method of Winter and co-coworkers (Jones et al (1986) Nature 321:522-525; Riechmann et al (1988) Nature 332:323-327: Verhoeyen et al (1988) Science 239: 1534-1536), by substituting rodent and mutant rodent CDRs or CDR sequences for the corresponding sequences of human antibody. In some instances, residues within the framework regions of one or more variable regions of the human immunoglobulin are replaced by corresponding non-human residues (see, for example, U.S. Pat. Nos. 5,585,089; 5,693,761; 5,693,762; and 6,180,370). Furthermore, humanized antibodies may comprise residues that are not found in the recipient antibody or in the donor antibody.

The antibodies of the invention may include amino acid residues not encoded by human sequences (e.g., mutations introduced by random or site-specific mutagenesis in vitro or by somatic mutation in vivo).

The term “recombinant antibody”, as used herein, includes all human or humanized antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human or humanized antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human or humanized antibodies have variable regions in which the framework and CDR regions may be derived from human germline immunoglobulin sequences. In certain embodiments, however, such recombinant human or humanized antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, the percent identity between the two sequences is a function of the number of identical positions shared by the sequences (i. e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Other sequence algorithms may alternatively be used such as BLAST, FASTA, using the default parameters for example.

The term “patient” refers to any subject (preferably human) afflicted with or susceptible to be afflicted with a thalassemia disorders.

“Treatment” is herein defined as the application or administration of an antagonist according to the invention, for example, an anti-TfR1 antibodies or a antigen-binding portion of said anti-TfR1 antibody as defined above, to a subject, or application or administration a pharmaceutical composition comprising said antagonists of the invention to an isolated tissue or cell line from a subject, where the subject has a thalassemia disorder, or a predisposition toward development of a thalassemia disorders, where the purpose is to cure, heal, alleviate, relieve, alter, remedy, ameliorate, improve, or affect the thalassemia disorder and/or any associated symptoms of the thalassemia disorder, or the predisposition toward the development of the thalassemia disorder.

By “treatment” is also intended the application or administration of a pharmaceutical composition comprising said agonists, to a subject, or application or administration of a pharmaceutical composition comprising said antagonists of the invention to an isolated tissue or cell line from a subject, where the subject has a thalassemia disorder, a symptom associated with a thalassemia disorder, or a predisposition toward development of a thalassemia disorder, where the purpose is to cure, heal, alleviate, relieve, alter, remedy, ameliorate, improve, or affect the a thalassemia disorder, any associated symptoms of the thalassemia disorder, or the predisposition toward the development of the a thalassemia disorder.

By “positive therapeutic response” with respect to a thalassemia disorder is intended an improvement in the disease in association with the correction of erythropoiesis activity of these molecules according to the invention, and/or an improvement in the symptoms associated with the disease.

By “therapeutically effective dose or amount” or “effective amount” is intended an amount of a agonists of the invention that, when administered brings about a positive therapeutic response with respect to treatment of a subject with an autoimmune disease and/or inflammatory disease. In some embodiments of the invention, a therapeutically effective dose of the antagonists of the invention, is in the range from 0.01 mg/kg to 100 mg/kg, from 0.1 mg/kg to 20 mg/kg. The method of treatment may comprise a single administration of a therapeutically effective dose or multiple administrations of a therapeutically effective dose of the agonists of the invention.

### Anti-TfR1 Antibodies According to the Invention or Antigen-Binding Portion Thereof

In one preferred embodiment, the antagonist according to the invention is an anti-TfR1 antibody or an antigen-binding portion thereof, which competitively inhibits binding of transferrin to TfR1.

The antibody or the antigen-binding portion thereof may be obtained by any well known method in the art. For example, the antibodies can be obtained using a variety of techniques, including conventional monoclonal antibody methodology, e.g., the standard somatic cell hybridoma technique of Kohler and Milstein, 1975, Nature, 256: 495. The hybridoma using the murine system is a well established procedure. Immunization protocols and techniques for isolation of immunized splenocytes for fusion are known in the art. Fusion partners (e.g., murine myeloma cells) and fusion procedures are also known. Such murine antibodies may then be humanized, for example by inserting the CDR regions into a human framework using methods known in the art. See e.g. U.S. Pat. No. 5,225,539 to Winter, and U.S. Pat. Nos. 5,530,101, 5,585,089, 5,693,762 and 6180370 to Queen et al.

In particular embodiment the antibody is a chimeric antibody (e.g. a human/mouse antibody) or a humanized antibody.

In a particular embodiment, the antibodies of the invention are human monoclonal antibodies. Such human monoclonal antibodies fulfilling the binding properties of the present invention can be identified using transgenic or transchromosomic mice carrying parts of the human immune system rather than the mouse system. These transgenic and transchromosomic mice include mice referred to herein as HuMab mice and KM mice, respectively, and are collectively referred to herein as “human Ig mice”. For example, the HuMAb mouse (Medarex, Inc) contains human immunoglobulin gene miniloci that encode un-rearranged human heavy (μ and γ) and κ light chain immunoglobulin sequences, together with targeted mutations that inactivate the μ and κ chain loci (see e.g. Lonberg, et al, 1994, Nature 368(6474): 856-859). Human recombinant antibodies can also be prepared using phage display methods for screening libraries of human immunoglobulin genes. Such phage display methods for isolating human antibodies with desired binding specificity are established in the art. See for example: U.S. Pat. Nos. 5,223,409, 5,403,484; and 5,571,698 to Ladner et al.; U.S. Pat. Nos. 5,427,908 and 5,580,717 to Dower et al; U.S. Pat. Nos. 6,544,731; 6,555,313; 6,582,915 and 6,593,081 to Griffiths et al.

In a particular embodiment, the anti-TfR1 antibody is TIB 220 antibody (ATCC catalog number: TIB-220 Clone Name: r17_{—}208.2) or a antigen binding portion of TIB220. In another particular embodiment, the antibody is a chimeric or humanized form of TIB220.

In another embodiment, the antagonist of TfR1 is selected from the group consisting of 42/6 antibody, 3TF12 antibody, 3GH7 antibody, IgG3-avidin fusion protein (ch128.1Av), that are disclosed in Daniels et al (Clinical Immunology (2006) 121, 144-158 and 159-176), Daniels et al (Biochimica et BhiophysicaActa 1820 (2012) 291-317), Crepin R et al (Cancer Research 70(13) 2010 5497-5506) and Brooks et al (Clinical Cancer Research: 1995 (1), 1259-1265) all of which are herein incorporated by reference. Antigen binding portion of the antibodies as previously mentioned are also encompassed in the present invention, as well as chimeric or humanized forms of said antibodies.

In a preferred particular embodiment, the antagonist of TfR1 is A24 antibody or an antigen-binding portion thereof. A24 antibody was described in WO2005111082. The hybridoma A24 secreting this antibody has been deposited, according to the terms of the Budapest Treaty, with the CNCM (Collection nationale de Cultures de Microorganismes,) on May 10 2001, under number 1-2665. Using surface plasmon resonance analysis, inventors showed that A24 antibody associates with TfR-1 and competes with Fe-Tf for receptor binding. They also determined that A24 antibody has a lower affinity to TfR-1 than Fe-Tf (2.69 versus 0.98 nM, respectively). However, under high receptor density; A24 binding to TfR-1 was higher than that of Fe-Tf due to avidity interactions of bivalent antibody. Thus, A24 can specifically particularly suitable for the treatment of thalassemia according to the invention.

In a particular embodiment, the antagonist of TfR1 is a chimeric or humanized form of A24.

In a particular embodiment, the antagonist of TfR1 is an antigen-binding portion of a chimeric or a humanized form of A24.

In one embodiment, the antagonist of TfR1 is antibody that binds to or competes for the same epitope as A24 More specifically, the invention relates to anti-TfR1 antibodies, or their antigen-binding portion, that binds to the epitope of A24, and that competitively inhibits binding of transferrin to transferrin receptor through binding to said epitope. For example, the invention antibodies may comprise a variable heavy chain (VH) and a variable light chain (VL) sequences where the CDR sequences share at least 60, 70, 90, 95 or 100 percent sequence identity to the corresponding CDR sequences of mAb A24, wherein said homologous antibody specifically binds to human TfR1, and the homologous antibody competitively inhibits binding of transferrin to transferrin receptor. Antibodies with mutant amino acid sequences can be obtained by mutagenesis (e.g., site-directed or PCR-mediated mutagenesis) of the coding nucleic acid molecules, followed by testing of the encoded altered antibody for retained function (i. e., the functions set forth above) using the functional assays described above. In certain embodiments, the homologous antibodies as described above have conservative sequence modifications compared to anti-TfR1 antibodies known as antagonists. As used herein, the term “conservative sequence modifications” is intended to refer to amino acid substitutions in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family, and the altered antibody can be tested for retained function using the functional assays described herein. Modifications can be introduced into an antibody of the invention by standard techniques known in the art, such as site-directed mutagenesis and PCR-mediated mutagenesis.

### Pharmaceutical Formulations and Modes of Administration

In another aspect the present invention provides a composition, e.g., a pharmaceutical composition, containing the antagonists of the present invention, formulated together with a pharmaceutically acceptable carrier.

Pharmaceutical formulations comprising the antagonists of the invention may be prepared for storage by mixing the antagonists, for example the anti-TfR1 antibodies or antigen-binding portion of said antibodies, having the desired degree of purity with optional physiologically acceptable carriers, excipients or stabilizers {Remington: the Science and Practice of Pharmacy 2Oth edition (2000)), in the form of aqueous solutions, lyophilized or other dried formulations. Therefore, the invention further relates to a lyophilized or liquid formulations comprising at least the anti-TfR1 antibodies or antigen-binding portion of said anti-TfR1 of the invention.

As used herein, ‘pharmaceutically acceptable carrier’ includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt’ refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic hydroiodic phosphorous and the like as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′ [symbol] dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate. alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

Alternatively, the antagonists of the invention can be administered as a sustained release formulation in which case less frequent administration is required. Dosage and frequency vary depending on the half-life of the compounds in the patient.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed. or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A composition of the present invention can be administered by one or more routes of administration using one or more of a variety of methods known in the art. As will be appreciated by the skilled artisan, the route and/or mode of administration will vary depending upon the desired results. Routes of administration for the agonists according to the invention include intravenous, intramuscular, intradermal, intraperitoneal, subcutaneous, spinal or other parenteral routes of administration, for example by injection or infusion. The phrase “parenteral administration” as used herein means modes of administration other than enteraI and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Alternatively, the agonists of the invention can be administered by a nonparenteral route, such as a topical, epidermal or mucosal route of administration, for example, intranasally, orally, vaginally, rectally, sublingually or topically.

Biodegradable, biocompatible polymers can be used for controlled release formulations, such as ethylene vinyl acetate, polyanhydrides, polyglycolic acid, collagen, polyorthoesters and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art.

The invention will be further illustrated by the following figures and examples. However, these examples and figures should not be interpreted in any way as limiting the scope of the present invention.

### FIGURES

FIG. 1: TIB-220 treatment reduced spleen, but not bone marrow, erythroblasts numbers in Hbb th1/th1 thalassemic mice. Hbb th1/th1 mice were treated for 60 days with TIB-220 or PBS. C57BL/6 mice were used as controls. Total bone marrow and spleen cell numbers (panels A and B) and Ter119+ erythroblasts (panels C and D) were evaluated

FIG. 2: TIB-220 treatment reduced bilirubin levels in Hbb th1/th1 thalassemic mice. Biochemical analysis of bilirubin levels in serum harvested from C57BL/6 or Hbb th1/th1 mice treated for 60 days with TIB-220 or PBS.

FIG. 3: In vivo TIB-220 treatment modulates erythropoiesis. Hbb th1/th1 thalassemic mice were treated for 60 days with TIB-220 or PBS. Spleen (upper panels) and bone marrow (lower panels) were harvested and erythroblast differentiation was evaluated by flow cytometry by CD71/TER-119 staining and FSC/SSC distribution. Pro-E: Proerythroblast, Ery-A: basophil Eryhtroblasts, Ery-B: late basophilic and polychromatic erythroblasts and Ery-C: orthochromatic erythroblasts and reticulocytes.

FIG. 4: Evaluation of hematological parameters of C57BL/6 mice and Hbb th1/th1 mice treated or not with TIB-220. Hbb th1/th1 thalassemic mice were treated for 60 days with TIB-220 or PBS. The effect of TIB-220 on hematological parameters was evaluated on day 60.

FIG. 5: TIB-220 treatment reduced Tf saturation and iron level but increased transferrin and ferritin levels. Biochemical analysis of serum harvested from WT C57BL/6 or Hbb th1/th1 mice treated for 60 days with TIB-220 or PBS.

### EXAMPLE

### Material & Methods Mice

Hbb (th1/th1) mice were used as a model of β-thalassemia intermedia. C57BL/6 mice were used as controls. All animals were housed under SPF conditions.

Treatment

Hbb (th1/th1) mice were treated twice a week with intraperitoneal (ip) injections of anti-transferrin receptor TIB-220 (10 mg/kg body weight during 60 days). PBS was used as control treatment.

Biological Parameters

Biological parameters were evaluated on day 60 in TIB-220 and PBS-treated Hbb (th1/th1) mice. Red blood cells (RBC), reticulocytes, mean corpuscular volume (MCV), hematocrit, hemoglobin (Hb), MHC and MHCH levels were evaluated with an MS5-9 automat.

Biochemical Parameters

Biochemical parameters were evaluated on day 60 in TIB-220 and PBS-treated Hbb (th1/th1) mice. Bilirubin (direct and total), transferrin, iron and ferritin were evaluated with a multiparametric automat Olympus AU400.

Immunofluorescence Analysis by Flow Cytometry

For the BM and splenocyte suspensions from mice, blocking of IgG receptors was performed with anti-FcγR mAb 2.4G2. Cells (1×106) were then stained with anti-TER-119 antibody and anti-mouse TfR1 antibody. Stained cells were analyzed by flow cytometry (FACScanto; Becton Dickinson). Data were analyzed with FlowJo software (Tree Star).

Statistical Analyses

Statistical analyses were performed with GraphPad Prism (version 5.0; GraphPad Software). The data are expressed as the mean±SEM of n determinations unless noted otherwise. Student's t-test or the Mann-Whitney test was used to compare two groups. Differences were considered significant at a P value less than 0.05 (*), less than 0.01 (**) or less than 0.001 (***).

Results

We sought to study the therapeutic effect of blocking TfR1 function in a mouse model of thalassemia intermedia (Hbbth1/th1 mice [9]) by using a well characterized anti-TfR1 monoclonal antibody (mAb TIB-220; rat IgM isotype) that blocks the uptake of iron-loaded transferrin by targeted cells [8]. Since IgM half-life is of poor in bloodstream (compared to the 23 days of IgG isotype) mice were treated twice a week [14, 15, 16].

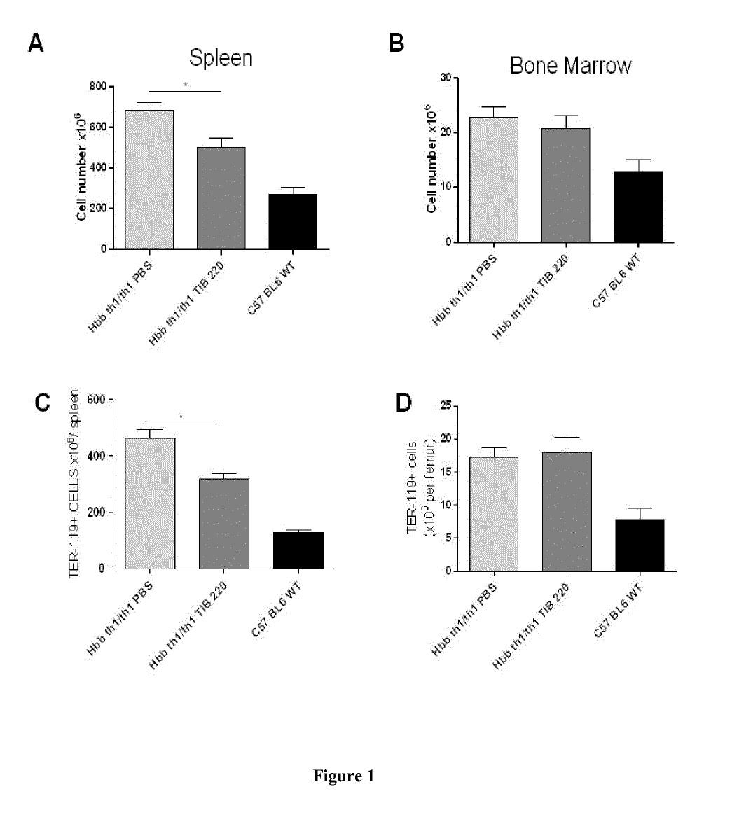

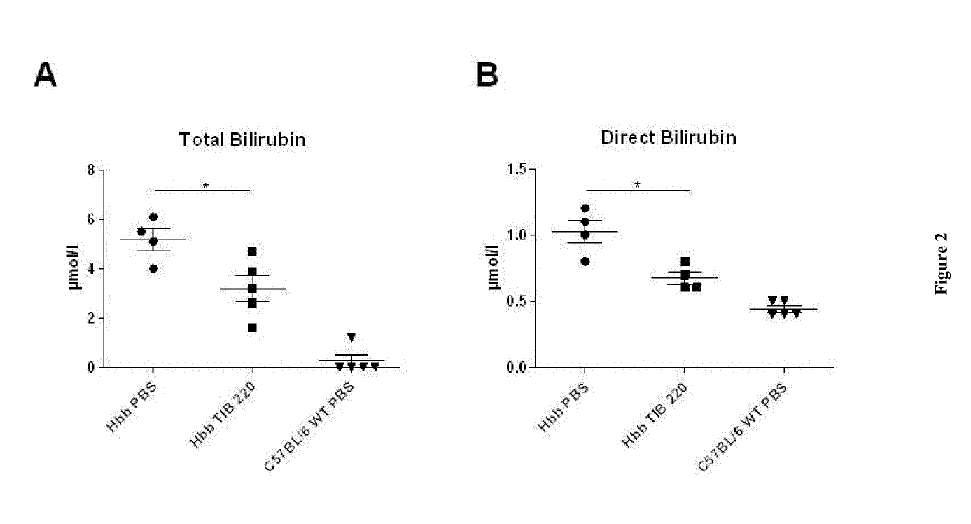

In thalassemia reduced RBC half-life due to the production of α-globin precipitates leads to anemia and the activation of stress compensatory mechanisms such as splenomegaly and hepatomegaly [17]. In agreement splenomegaly was observed in thalassemic Hbbth1/th1 mice which presented around 3 fold increase in spleen cell numbers compared to C57BL6 control mice (FIG. 1A) and as previously described [7]). However, in mice treated with anti-TfR1 antibodies for 60 days there was reduction in spleen weight and spleen erythroblasts numbers (Ter119+ cells) compared to mock treated mice (FIG. 1A-C). Interestingly bone marrow erythroblasts numbers were not reduced in anti-TfR1 treated mice suggesting that spleen erythroblasts are more sensitive to cellular iron uptake impairment than bone marrow erythroblasts (FIG. 1B-D). Accordingly total bilirubin and direct bilirubin levels where normalized in anti-TfR1 treated animals further suggesting that tissue hemolysis was decreased in treated animals (FIG. 2). Therefore anti-TfR1 therapy partially reversed splenomegaly observed in thalassemic mice.

In thalassemia the overall stress erythropoiesis caused by anemia leads to increased proliferation of immature erythroid progenitors, an accelerated erythroid differentiation and the accumulation of polychromatophil erythroblasts [18,19]. There is also an increased apoptosis of maturing erythroblasts due to α-globin precipitation in cell membranes leading to ineffective erythropoiesis [19]. In mice, spleen is the main site committed to stress erythropoiesis whereas bone marrow is mainly implicated in the maintenance of steady-state erythropoiesis [20]. We therefore searched to evaluate whether blocking TfR1 function would impact on erythroid differentiation in tissues committed to steady-steate and stress erythropoiesis.

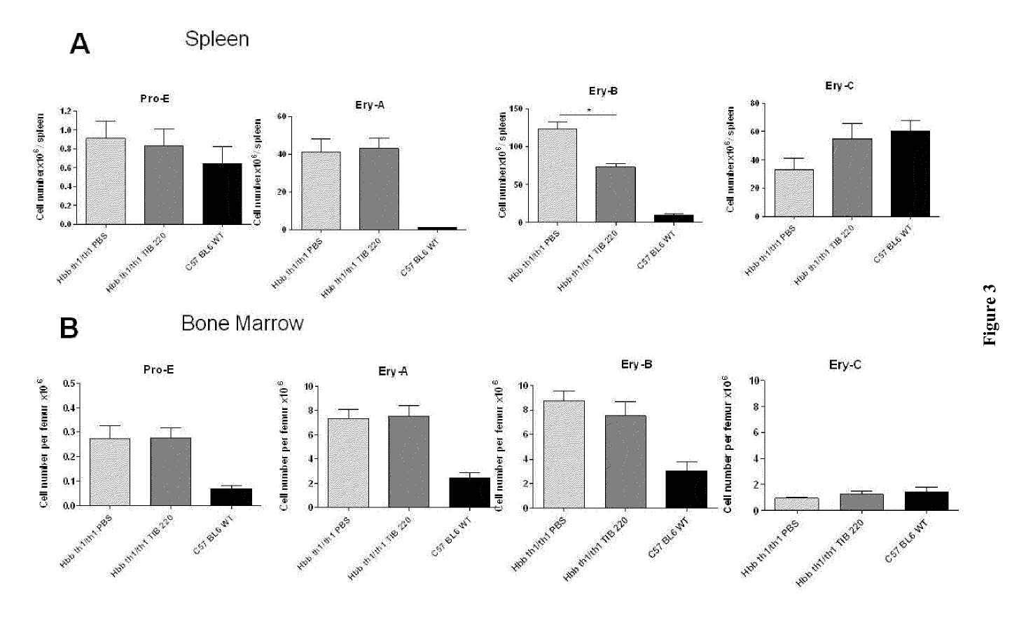

Flow cytometry analysis of different erythroblast populations [21] in spleen show that late basophilic and polychromatic erythroblasts (Ery B) were decreased in anti-TfR1 treated mice (FIG. 3A). These changes were followed by an increase in orthochromatic erythroblasts and reticulocytes (Ery C) (FIG. 3A). Accordingly, with the data previously described for total erythroblasts, anti-TfR1 antibodies did not interfere on steady-steate erythopoieis since there was no difference in erythroid cell populations in the bone marrow of treated and untreated animals (FIG. 3B). Altogether these data suggest that anti-TfR1 antibodies blocked spleen stress erythropoiesis and reversed IE of β-thalassemia.

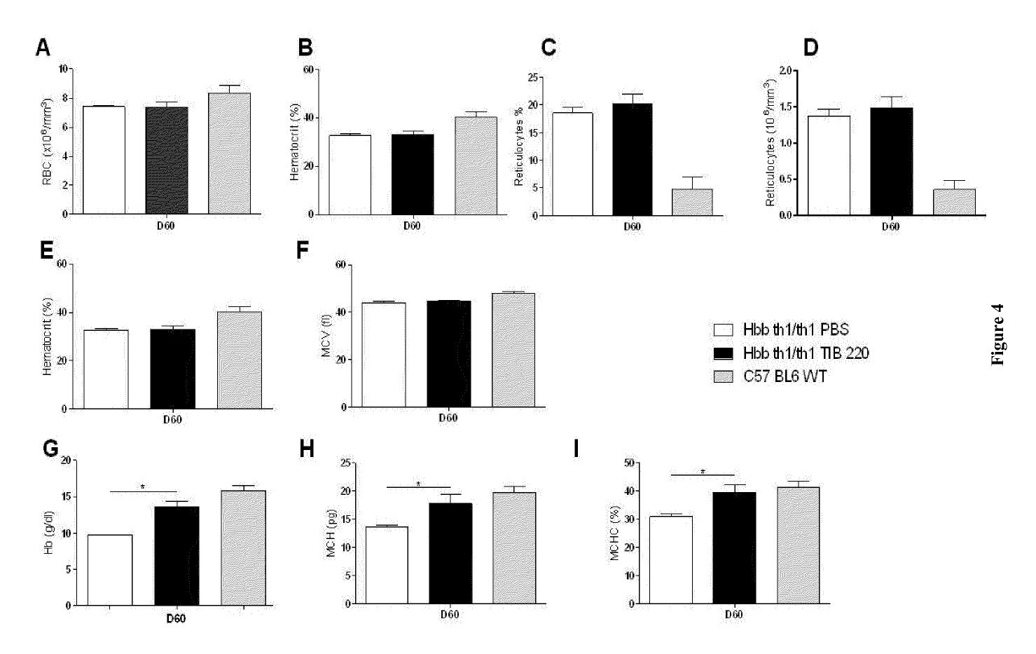

We therefore searched to evaluate the impact of changes in stress erythropoiesis in blood parameters. Red blood cells (RBC) and reticulocytes numbers as well as hematocrit and mean corpuscular volume (MCV) did not differ between treated and untreated mice (FIG. 4A-F). However, there was a significant increase in hemoglobin levels and Mean corpuscular hemoglobin (MCH) and MCH concentration (MCHC) (FIG. 4G-I) in treated mice. Therefore anti-TfR1 impact in erythropoiesis induced the production of RBC with increased hemoglobin content and ameliorated the anemia in anti-TfR1 treated animals.

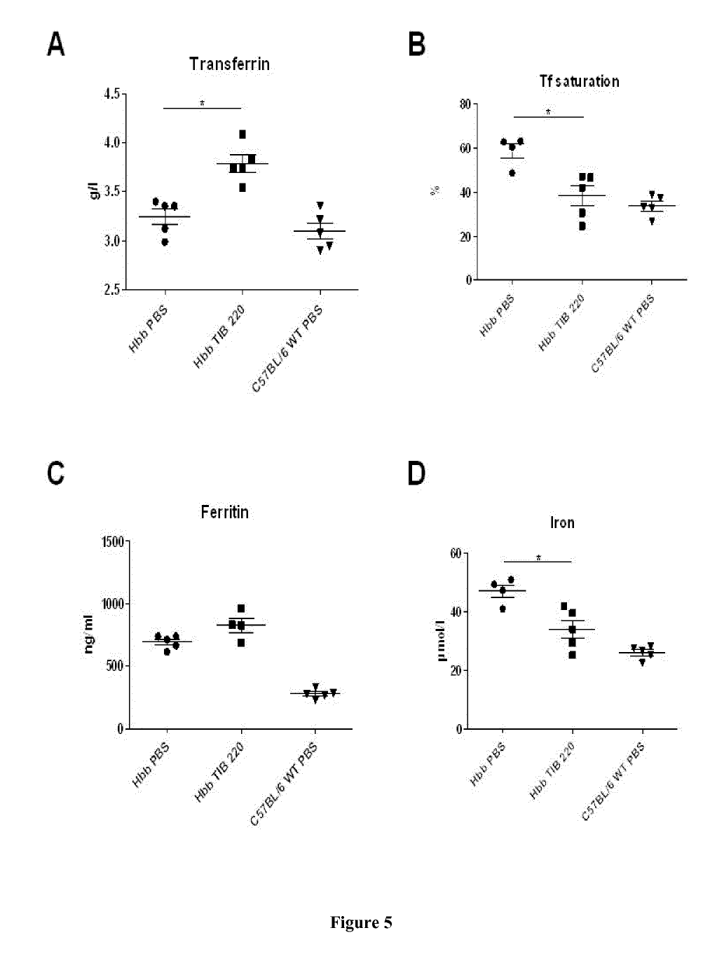

Since iron overload occurs in β-thalassemia and TfR1 is the main receptor implicated in cellular iron uptake which consequently would impact on iron homeostasis we examined the consequences of anti-TfR1 in parameters of iron homeostasis. Serum transferrin levels were increased in anti-TfR1 treated animals compared to mock treated animals (FIG. 5A). Increased transferrin levels lead to a complete normalization of transferrin saturation in the serum of treated animals (FIG. 5B). There was no differences in serum ferritin levels between treated and control mice (FIG. 5C). However there was a significant decrease in serum iron levels further suggesting that anti-TfR1 antibodies decreased serum iron parameters in β-thalassemic mice (FIG. 5D).

Altogether the data presented here shows the safety and efficacy of impairing TfR1 function in an experimental model β-thalassemia. In mice, homeostatic steady state erythopoiesis is mainly supported by bone marrow erythroblasts whereas stress erythopoiesis is dependent on the proliferation of spleen stress erythroblasts in response to specific signals including, Hedgehog, hypoxia, SCF and BMP4 [20]. Stress erythopoiesis frequently occurs in acute conditions (such as anemia, adaptation to higher altitudes) but in hemolytic and iron overloading anemia there is a chronic activation of spleen stress erythropoiesis that culminates with splenomegaly [19]. Ineffective erythorpoiesis (IE) is a particular condition in β-thalassemia where stress responses triggered by anemia induce an increased proliferation and an accelerated differentiation of early stage erythroblasts. However these primed maturing stress erythroblasts will finish to accumulate α-globin precipitates and die from apoptosis. This ineffective process results in a massive tissue hemolysis. In addition to decreased red blood cell (RBC) production a shortened red blood cell (RBC) survival also contribute to moderate-to-severe anemia in β-thalassemia and the maintenance of chronic IE condition. Targeting of TfR1 greatly reduced the numbers of stress polychromatophil erythroblasts in the spleen and serum bilirubin levels further suggesting that tissue hemolysis and IE was decreased in treated animals.

Transferrin receptor is the major receptor implicated in iron uptake to guarantee hemoglobin synthesis and TfR1 knockout mice are anemic and dye at E12.5 [22]. Therefore anemia is expected to be a major complication of anti-TfR1 therapy. However, our data suggest that in contrast to stress erythropoiesis steady-state erythopoiesis is not affected by anti-TfR1 therapy. In agreement, treated mice did not become anemic despite of antibody treatment of 60 days. These data suggests that requirement of iron uptake in steady state and stress erythopoiesis would be different. In addition, anti-TfR1 treated mice increased hemoglobin levels without increased of RBC numbers therefore paradoxically increasing hemoglobin content/RBC (MCH and MCHC). These data suggest that iron overload in thalassemic erythroblasts would lead to the production of RBC with a decreased hemoglobin content.

Thalassemic patients usually require RBC transfusions to reduce anemia and therefore guarantee adequate tissue oxygen delivery. Thalassemic patients also present increased intestinal iron absorption which associated with transfusional iron loading lead to increased transferrin saturation and appearance of non-transferrin bound iron (NTBI) which finishes accumulating in the parenchyma of several tissues. Here, impairment of TfR1 function by anti-TfR1 blocking antibodies lead to an increased production of transferrin. Increased Tf levels lead to a reduction in transferrin saturation and would be an alternative to treat patients with increased amounts of NTBI. TfR1 blocking also reduced serum iron levels. Therefore, tissue damage caused by parenchymal iron overload could be prevented by blocking TfR1 function. Finally we also believe that anti-TfR1 therapy would contribute to decrease the time length where thalassemic patients would be transfusion independent therefore reducing the need of regular transfusions which contribute to iron overload.

Altogether, the data presented here show that impairing TfR1 function decreased the main causes of pathology in β-thalassemia named ineffective erythropoiesis, tissue hemolysis and iron overload. Therefore, blocking TfR1 function is an alternative therapeutic target in this disease.

### REFERENCES

Throughout this application, various references describe the state of the art to which this invention pertains. The disclosures of these references are hereby incorporated by reference into the present disclosure.
1. Higgs D R, Engel J D, Stamatoyannopoulos G (2012) Thalassaemia. Lancet 379: 373-383.
2. Rachmilewitz E A, Weizer-Stern O, Adamsky K, Amariglio N, Rechavi G, et al. (2005) Role of iron in inducing oxidative stress in thalassemia: Can it be prevented by inhibition of absorption and by antioxidants? Ann N Y Acad Sci 1054: 118-123.
3. Kihm A J, Kong Y, Hong W, Russell J E, Rouda S, et al. (2002) An abundant erythroid protein that stabilizes free alpha-haemoglobin. Nature 417: 758-763.
4. Sorensen S, Rubin E, Polster H, Mohandas N, Schrier S (1990) The role of membrane skeletal-associated alpha-globin in the pathophysiology of beta-thalassemia. Blood 75: 1333-1336.
5. Rivella S (2012) The role of ineffective erythropoiesis in non-transfusion-dependent thalassemia. Blood Rev 26 Suppl 1: S12-15.
6. Gardenghi S, Ramos P, Marongiu M F, Melchiori L, Breda L, et al. (2010) Hepcidin as a therapeutic tool to limit iron overload and improve anemia in beta-thalassemic mice. J Clin Invest 120: 4466-4477.
7. Li H, Rybicki A C, Suzuka S M, von Bonsdorff L, Breuer W, et al. (2010) Transferrin therapy ameliorates disease in beta-thalassemic mice. Nat Med 16: 177-182.
8. Trowbridge I S, Lesley J, Schulte R (1982) Murine cell surface transferrin receptor: studies with an anti-receptor monoclonal antibody. J Cell Physiol 112: 403-410.
9. Skow L C, Burkhart B A, Johnson F M, Popp R A, Popp D M, et al. (1983) A mouse model for beta-thalassemia. Cell 34: 1043-1052.
10. Lambert L A (2012) Molecular evolution of the transferrin family and associated receptors. Biochim Biophys Acta 1820: 244-255.
11. Lambert L A, Mitchell S L (2007) Molecular evolution of the transferrin receptor/glutamate carboxypeptidase II family. J Mol Evol 64: 113-128.
12. Zhou X, Babu J R, da Silva S, Shu Q, Graef I A, et al. (2007) Unc-51-like kinase 1/2-mediated endocytic processes regulate filopodia extension and branching of sensory axons. Proc Natl Acad Sci USA 104: 5842-5847.
13. Doyon J B, Zeitler B, Cheng J, Cheng A T, Cherone J M, et al. (2011) Rapid and efficient clathrin-mediated endocytosis revealed in genome-edited mammalian cells. Nat Cell Biol 13: 331-337.
14. Haisma H J, Kessel M A, Silva C, van Muijen M, Roos J C, et al. (1990) Human IgM monoclonal antibody 16.88: pharmacokinetics and distribution in mouse and man. Br J Cancer Suppl 10: 40-43.
15. Mould D R, Sweeney K R (2007) The pharmacokinetics and pharmacodynamics of monoclonal antibodies—mechanistic modeling applied to drug development. Curr Opin Drug Discov Devel 10: 84-96.
16. Reichert J M, Dewitz M C (2006) Anti-infective monoclonal antibodies: perils and promise of development. Nat Rev Drug Discov 5: 191-195.
17. May C, Rivella S, Callegari J, Heller G, Gaensler K M, et al. (2000) Therapeutic haemoglobin synthesis in beta-thalassaemic mice expressing lentivirus-encoded human beta-globin. Nature 406: 82-86.
18. Rivella S, Rachmilewitz E (2009) Future alternative therapies for beta-thalassemia. Expert Rev Hematol 2: 685.
19. Ginzburg Y, Rivella S (2011) beta-thalassemia: a model for elucidating the dynamic regulation of ineffective erythropoiesis and iron metabolism. Blood 118: 4321-4330.
20. Paulson R F, Shi L, Wu D C (2011) Stress erythropoiesis: new signals and new stress progenitor cells. Curr Opin Hematol 18: 139-145.
21. Liu Y, Pop R, Sadegh C, Brugnara C, Haase V H, et al. (2006) Suppression of Fas-FasL coexpression by erythropoietin mediates erythroblast expansion during the erythropoietic stress response in vivo. Blood 108: 123-133.
22. Levy J E, Jin O, Fujiwara Y, Kuo F, Andrews N C (1999) Transferrin receptor is necessary for development of erythrocytes and the nervous system. Nat Genet 21: 396-399.

1. A method of treating a thalassemia disorder in a subject in need thereof, comprising
administering to said subject a therapeutically effective amount of an antagonist of transferrin receptor (TfR1).

2. The method according to claim 1 wherein said thalassemia disorder is β-thalassemia.

3. The method of claim 1, wherein said isolated antagonist of TfR1 is selected from the group consisting of:
i. an isolated anti-TfR1 antibody; and
ii. an antigen-binding portion of said anti-TfR1 antibody;
wherein said antibody or antigen binding portion thereof competitively inhibits binding of transferrin to transferrin receptor.

4. The method of claim 1 wherein said isolated antagonist of TfR1 is A24 antibody or an antigen-binding portion of A24 antibody.

5. The method of claim 1 wherein said isolated antagonist of TfR1 is a chimeric or humanized form of A24 or an antigen-binding portion of a chimeric or a humanized form of A24.

6. The method of claim 1 wherein said isolated antagonist of TfR1 is an isolated anti-TfR1 antibody or an antigen-binding portion of said anti-TfR1 antibody that competes for the same epitope as A24 antibody.

7. A pharmaceutical composition for use in treating a thalassemia disorder, comprising
an antagonist of TfR1 and
at least a pharmaceutically acceptable excipient, diluent or carrier.

8. The pharmaceutical composition of claim 7, additionally comprising at least one other active ingredient.

9. The pharmaceutical composition of claim 7 wherein said isolated antagonist of TfR1 is selected from the group consisting of:
i. an isolated anti-TfR1 antibody;
ii. an antigen-binding portion of said anti-TfR1 antibody;
wherein said antibody or antigen binding portion thereof competitively inhibits binding of transferrin to transferrin receptor.

10. The pharmaceutical composition of claim 7 wherein said isolated antagonist of TfR1 is A24 antibody or an antigen-binding portion of A24 antibody.

11. The pharmaceutical composition of claim 7 wherein said isolated antagonist of TfR1 is a chimeric or humanized form of A24 or an antigen-binding portion of a chimeric or a humanized form of A24.

12. The pharmaceutical composition of claim 7 wherein said isolated antagonist of TfR1 is an isolated anti-TfR1 antibody or an antigen-binding portion of said anti-TfR1 antibody that competes for the same epitope as A24 antibody.
